(19) United States
(12) Patent Application Publication
(10) Pub. No.: US 2009/0254080 A1
(43) Pub. Date: Oct. 8, 2009

(54) SURGICAL OPERATION APPARATUS

(76) Inventor: Satoshi HONDA, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 12/098,830

(22) Filed: Apr. 7, 2008

(51) Int. Cl. A61B 18/14

(52) U.S. Cl. 606/38

(57) ABSTRACT

A surgical operation apparatus includes a handpiece to be held by an operator, and an apparatus main body to be connected with the handpiece, wherein the handpiece includes a high-frequency treatment unit to treat a living tissue by a high-frequency current, an ultrasonic treatment unit to treat a living tissue by an ultrasonic vibration, and a sensor unit to perform detection for calculation of an impedance of a living tissue, and the apparatus main body includes a high-frequency output module to activate the high-frequency treatment unit, an ultrasonic output module to activate the ultrasonic treatment unit, a sensor module to calculate the impedance of the living tissue based on a detection result obtained from the sensor unit, and a control module to automatically control at least the ultrasonic output module of the high-frequency output module and the ultrasonic output module in accordance with the impedance calculated by the sensor module so as to automatically adjust an output of at least the ultrasonic treatment unit of the high-frequency treatment unit and the ultrasonic treatment unit.

### BACKGROUND OF THE INVENTION

1. Field of the Invention

The present invention relates to a surgical operation apparatus to apply a coagulation-incision treatment to a living tissue by both an ultrasonic vibration and a high-frequency current.

2. Description of the Related Art

Jpn. Pat. Appln. KOKAI Publication No. 6-42893 discloses a surgical apparatus to treat a living tissue by both an ultrasonic vibration and a high-frequency current. That is, in this surgical apparatus, a tool is coupled with a vibration system in a handpiece to be held by an operator. The handpiece is connected with a device to supply an ultrasonic energy through a cable. When the ultrasonic energy is supplied to the vibration system, ultrasonic vibration is generated in the vibration system and the tool is ultrasonically vibrated and brought into contact with a living tissue, the living tissue is fractured. On the other hand, the handpiece is connected with an electrosurgical unit to supply high-frequency energy through a cautery cable. When the tool is brought into contact with the living tissue, a high-frequency voltage is applied to the tool by the electrosurgical unit and the high-frequency current flows between the tool and a dispersed ground pad arranged outside a body, the living tissue is coagulated and incised.

Jpn. Pat. Appln. KOKAI Publication No. 2003-33369 also discloses a similar operation apparatus. In this operation apparatus, ultrasonic vibration and a high-frequency current can be simultaneously output, and when an output ratio adjustment knob in an adjustment panel is operated manually, a ratio of output of the ultrasonic vibration and output of the high-frequency current is adjusted.

The specification of U.S. Pat. No. 6,398,779 discloses a blood vessel sealing system to treat a living tissue by a high-frequency current. In this blood vessel sealing system, an impedance of a living tissue is measured, and output of the high-frequency current is adjusted based on this impedance.

### BRIEF SUMMARY OF THE INVENTION

In an aspect of the present invention, a surgical operation apparatus includes: a handpiece to be held by an operator; and an apparatus main body to be connected with the handpiece, wherein the handpiece includes: a high-frequency treatment unit to treat a living tissue by a high-frequency current; an ultrasonic treatment unit to treat a living tissue by an ultrasonic vibration; and a sensor unit to perform detection for calculation of an impedance of a living tissue, and the apparatus main body includes: a high-frequency output module to activate the high-frequency treatment unit; an ultrasonic output module to activate the ultrasonic treatment unit; a sensor module to calculate the impedance of the living tissue based on a detection result obtained from the sensor unit; and a control module to automatically control at least the ultrasonic output module of the high-frequency output module and the ultrasonic output module in accordance with the impedance calculated by the sensor module so as to automatically adjust an output of at least the ultrasonic treatment unit of the high-frequency treatment unit and the ultrasonic treatment unit.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view showing a surgical operation apparatus according to a first embodiment of the present invention;

FIG. 2 is a block diagram showing the surgical operation apparatus according to the first embodiment of the present invention;

FIG. 3 is a view showing a relationship between a change in a state of a living tissue and a change in an impedance;

FIG. 4 is a flowchart showing an automatic adjustment method for an output in the surgical operation apparatus according to the first embodiment of the present invention;

FIG. 5 is a timing chart of an output in the surgical operation apparatus according to the first embodiment of the present invention;

FIG. 6 is a flowchart showing an automatic adjustment method for an output in a surgical operation apparatus according to a second embodiment of the present invention;

FIG. 7 is a timing chart of an output in the surgical operation apparatus according to the second embodiment of the present invention;

FIG. 8 is a flowchart showing an automatic adjustment method for an output in a surgical operation apparatus according to a third embodiment of the present invention;

FIG. 9 is a timing chart of an output in the surgical operation apparatus according to the third embodiment of the present invention;

FIG. 10 is a flowchart showing an automatic adjustment method for an output in a surgical operation apparatus according to a fourth embodiment of the present invention; and

FIG. 11 is a timing chart of an output in the surgical operation apparatus according to the fourth embodiment of the present invention.

### DETAILED DESCRIPTION OF THE INVENTION

Each embodiment according to the present invention will now be explained hereinafter with reference to the drawings.

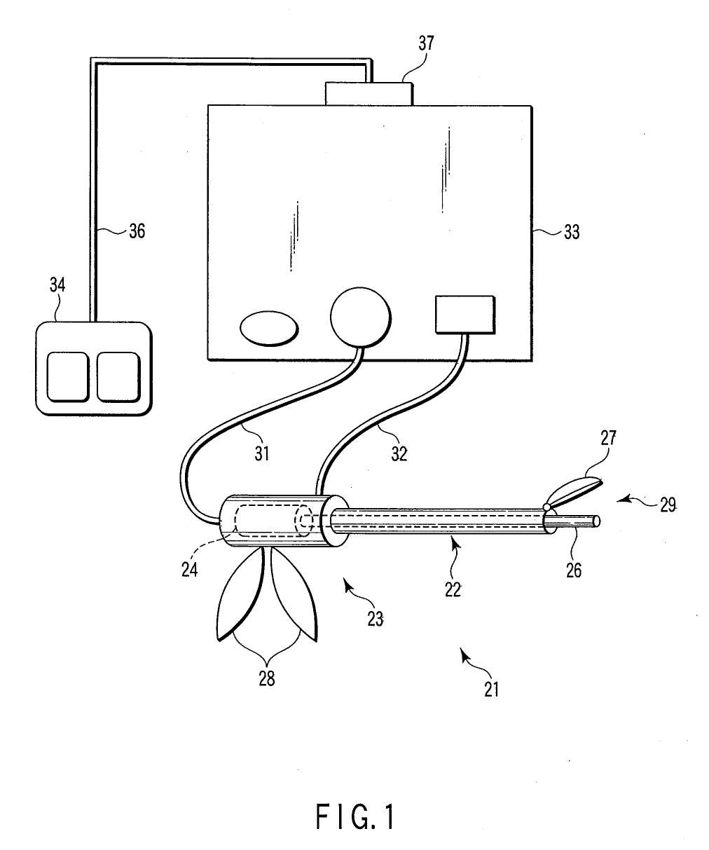

FIGS. 1 to 5 show a first embodiment according to the present invention.

A surgical operation apparatus according to this embodiment will now be explained with reference to FIG. 1.

The surgical operation apparatus includes a handpiece 21 to be held by an operator. In the handpiece 21, an operating section 23 is coupled with a proximal end portion of an elongated sheath 22.

An ultrasonic vibrator 24 is provided within the operating section 23. A proximal end portion of a probe 26 is coupled with the ultrasonic vibrator 24, the probe 26 is inserted into the sheath 22, and a distal end portion of the probe 26 protrudes from a distal end opening of the sheath 22. Ultrasonic vibration generated by the ultrasonic vibrator 24 is transmitted by the probe 26, and the distal end portion of the probe 26 is ultrasonically vibrated. A jaw 27 is arranged at the distal end portion of the sheath 22 and the jaw 27 is to be opened/closed with respect to the distal end portion of the probe 26 and grasp a living tissue in cooperation with the distal end portion of the probe 26. A grasping surface of the jaw 27 is covered with, e.g., a Teflon pad. A pair of handles 28 to open and close the jaw 27 is arranged in the operating section 23. That is, the distal end portion of the probe 26 and the jaw 27 form a grasping portion 29 to grasp the living tissue. When a living tissue is grasped by the grasping portion 29 and the distal end portion of the probe 26 is ultrasonically vibrated, the living tissue is treated. In this manner, the ultrasonic vibrator 24, the probe 26, and the jaw 27 form an ultrasonic treatment unit to treat the living tissue by the ultrasonic vibration. A treatment by the ultrasonic vibration has a high incision capability, and the incision capability is increased when an output of the ultrasonic treatment unit becomes large.

Further, the distal end portion of the probe 26 and the jaw 27 function as bipolar electrodes. That is, a high-frequency voltage can be applied between the distal end portion of the probe 26 and the jaw 27. When the living tissue is grasped by the grasping portion 29, the high-frequency voltage is applied between the distal end portion of the probe 26 and the jaw 27 and high-frequency current flows through the grasped living tissue, the living tissue is treated. In this manner, the grasping portion 29 forms a high-frequency treatment unit to treat the living tissue by the high-frequency current. A treatment through the bipolar electrodes by the high-frequency current has a high coagulation capability, and the coagulation capability is increased when an output of the high-frequency treatment unit becomes large.

Furthermore, an impedance of the living tissue grasped by the grasping portion 29 is calculated based on the high-frequency voltage applied between the distal end portion of the probe 26 and the jaw 27 and the high-frequency current flowing through the grasped living tissue. That is, the high-frequency treatment unit also serves as a sensor unit to perform detection for calculation of the impedance of the living tissue.

An ultrasonic handpiece cable 31 connected with the ultrasonic vibrator 24 is extended from the operating section 23 of the handpiece 21. Moreover, a high-frequency handpiece cable 32 connected with the probe 26 and the jaw 27 is extended from the operating section 23 of the handpiece 21. The ultrasonic handpiece cable 31 and the high-frequency handpiece cable 32 are connected with an apparatus main body 33.

On the other hand, the surgical operation apparatus includes a foot switch 34 to perform an output operation. A foot switch cable 36 is extended from the foot switch 34, and a foot switch connector 37 at an extended end portion of the foot switch cable 36 is detachably connected with the apparatus main body 33.

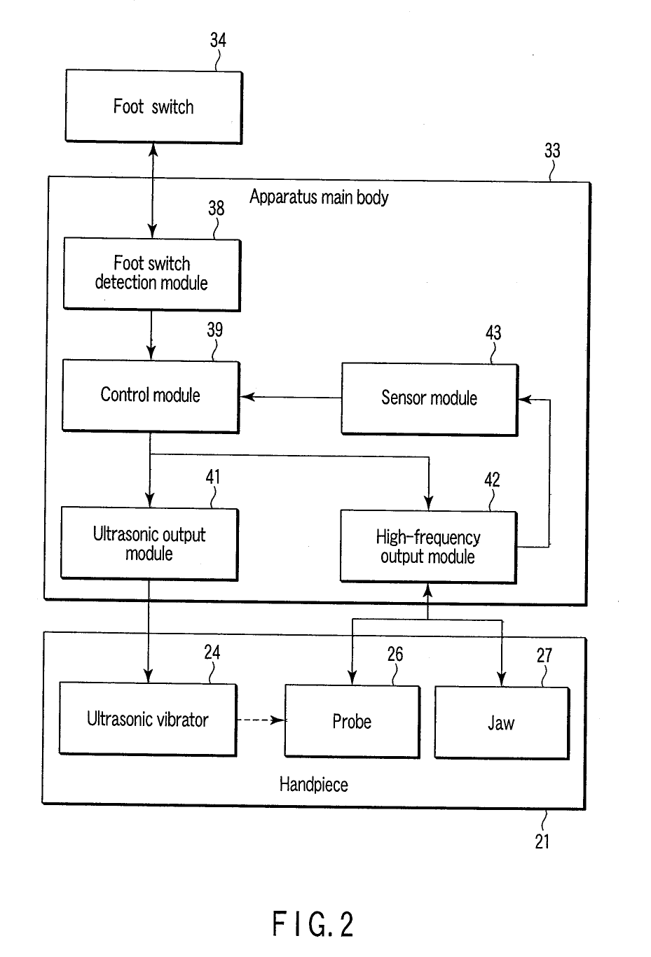

An automatic control system for an output in the surgical operation apparatus according to this embodiment will now be explained with reference to FIG. 2.

A foot switch detection module 38 in the apparatus main body 33 detects ON/OFF of the foot switch 34, and transmits a foot switch detection signal to a control module 39. As the foot switch detection signal, a foot switch ON signal indicating that the foot switch 34 is in an ON state and a foot switch OFF signal indicating that the foot switch 34 is in an OFF state is used.

The control module 39 transmits an ultrasonic control signal to an ultrasonic output module 41 based on the foot switch detection signal and controls the ultrasonic output module 41. The ultrasonic output module 41 actuates the ultrasonic vibrator 24 based on the ultrasonic control signal. As the ultrasonic control signal, an ultrasonic maximum output signal to actuate the ultrasonic vibrator 24 with a maximum output, an ultrasonic low output signal to actuate the same with a low output, an ultrasonic micro output signal to actuate the same with a micro output, and an ultrasonic stop signal to stop the ultrasonic vibrator 24 is used. Here, when the ultrasonic vibrator 24 is actuated with the maximum output, the low output, or the micro output, an incision capability becomes maximum, relatively low, or substantially zero. In this manner, the ultrasonic treatment unit is actuated with the maximum output, the low output, or the micro output, or stopped.

Moreover, the control module 39 transmits a high-frequency control signal to a high-frequency output module 42 to control the high-frequency output module 42 based on the foot switch detection signal. The high-frequency output module 42 supplies a high-frequency current to the grasping portion 29 based on the high-frequency control signal. As the high-frequency control signal, a high-frequency regular output signal that supplies a high-frequency current as a regular output to the grasping portion 29, a high-frequency micro output signal that supplies a high-frequency current as a micro output to the same, and a high-frequency stop signal that stops the high-frequency current is used. Here, when the high-frequency current as the regular output or the micro output is supplied to the grasping portion 29, a coagulation capability becomes normal or substantially zero. In this manner, the high-frequency treatment unit is actuated with the regular output or the micro output, or stopped.

Additionally, the high-frequency voltage applied and the high-frequency current supplied from the high-frequency output module 42 to the grasping portion 29 are measured by a sensor module 43. The sensor module 43 calculates an impedance of the living tissue grasped by the grasping portion 29 based on the measured high-frequency voltage and high-frequency current, and transmits impedance data to the control module 39.

The control module 39 controls the ultrasonic output module 41 and the high-frequency output module 42 based on the transmitted impedance data and adjusts outputs of the ultrasonic treatment unit and the high-frequency unit.

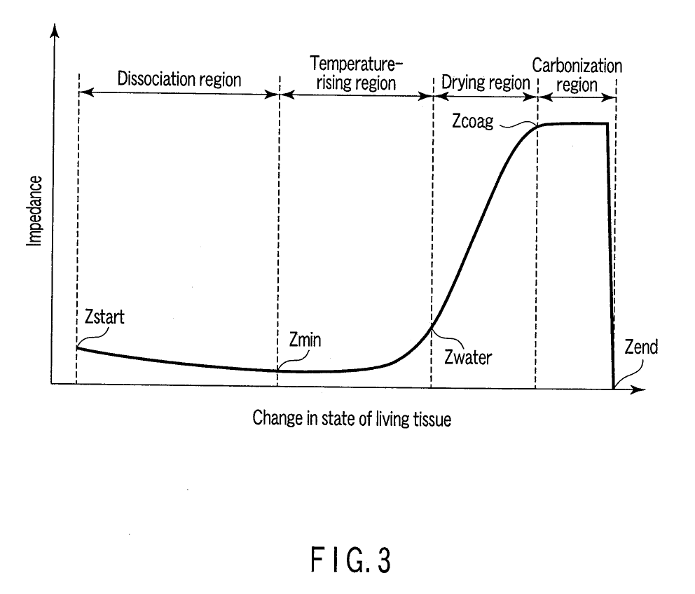

A relationship between a change in a state of the living tissue and a change in the impedance will now be explained with reference to FIG. 3.

At the start of a flow of the high-frequency current, the impedance of the living tissue indicates a certain initial value Zstart. As the high-frequency current flows through the living tissue, salt contained in a humor of the living tissue is dissociated. This region will be referred to as a dissociation region. In the dissociation region, the impedance is gradually reduced, and starts increasing when dissociation is completed. A value of the minimum impedance indicative of completion of dissociation will be referred to as a minimum value Zmin. Then, a temperature of moisture, e.g., a humor of the living tissue rises. This region will be referred to as a temperature-rising region. In this region, the impedance is gradually increased from the minimum impedance. Subsequently, the moisture in the living tissue boils and evaporates, and the living tissue is dried. This region will be referred to as a drying region. With start of drying of the living tissue, a rate of change of the impedance is suddenly increased. A value of the impedance which indicates start of drying of the living tissue and with which the rate of change is suddenly increased will be referred to as a drying start value Zwater. Here, with start of drying of the living tissue, elasticity of the living tissue begins to disappear, and the living tissue starts contracting. That is, the drying start value Zwater is an elasticity disappearance start value Zwater indicating that the elasticity of the living tissue has started to disappear, and a contraction start value indicating that the living tissue has started to contract. Then, the moisture in the living tissues disappears, and the living tissue begins carbonization. It can be determined that coagulation of the living tissue is completed at this time point. With completion of coagulation of the living tissue, the rate of change in the impedance becomes substantially zero, and the impedance has a relatively high fixed value. The value of the relatively high fixed impedance which indicates completion of coagulation of the living tissue and with which the rate of change becomes substantially zero will be referred to as a coagulation completion value Zcoag. Then, carbonization of the living tissue progresses. This region will be referred to as a carbonization region. In the carbonization region, the value of the impedance is held at the coagulation completion value Zcoag. Thereafter, when incision of the living tissue by the probe 26 of the ultrasonic treatment unit is completed and the jaw 27 and the probe 26 of the high-frequency treatment unit come into contact with each other to be short-circuited, the impedance becomes zero. The value of the impedance which indicates completion of incision and becomes zero will be referred to as an incision completion value Zend.

A description will now be given as to a detection method for the minimum value Zmin, the drying start value Zwater, the coagulation completion value Zcoag, and the incision completion value Zend by the control module 39.

In regard to the minimum value Zmin, when the impedance is reduced from the initial value Zstart and becomes a minimal value, the control module 39 detects that the impedance has the minimum value Zmin. As to the drying start value Zwater, the drying start value Zwater is larger than the initial value Zstart and, when the impedance is increased from the minimum value Zmin and suddenly increased and the rate of change of the impedance exceeds a certain threshold value, the control module 39 detects that the impedance has the drying start value Zwater. Further, the drying start value Zwater may be determined from at least one of the initial value Zstart and the minimum value Zmin. In regard to the coagulation completion value Zcoag, the coagulation completion value Zcoag is larger than the initial value Zstart and, when the impedance is increased from the drying start value Zwater, this increase becomes gentle, the rate of change of impedance becomes a certain threshold value or below, and the impedance takes a fixed value, the control module 39 determines that the impedance has the coagulation completion value Zcoag. Furthermore, the coagulation completion value Zcoag may be determined from at least one of the initial value Zstart, the minimum value Zmin, and the drying start value Zwater. As to the incision completion value Zend, when the impedance becomes zero, the control module 39 detects that the impedance has the incision completion value Zend.

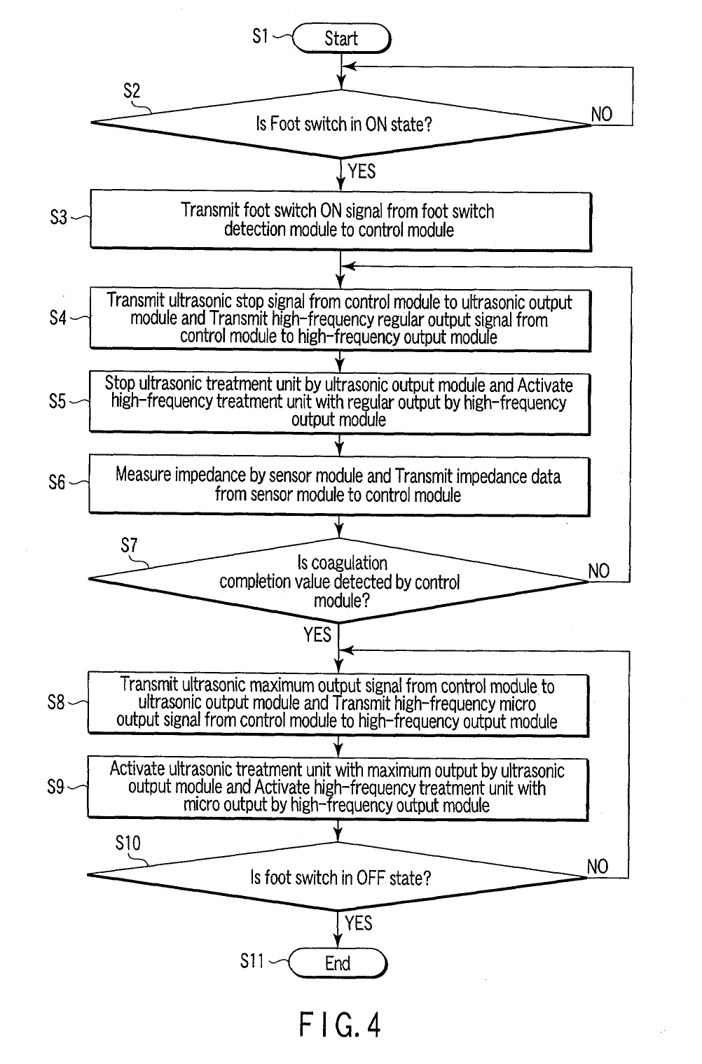

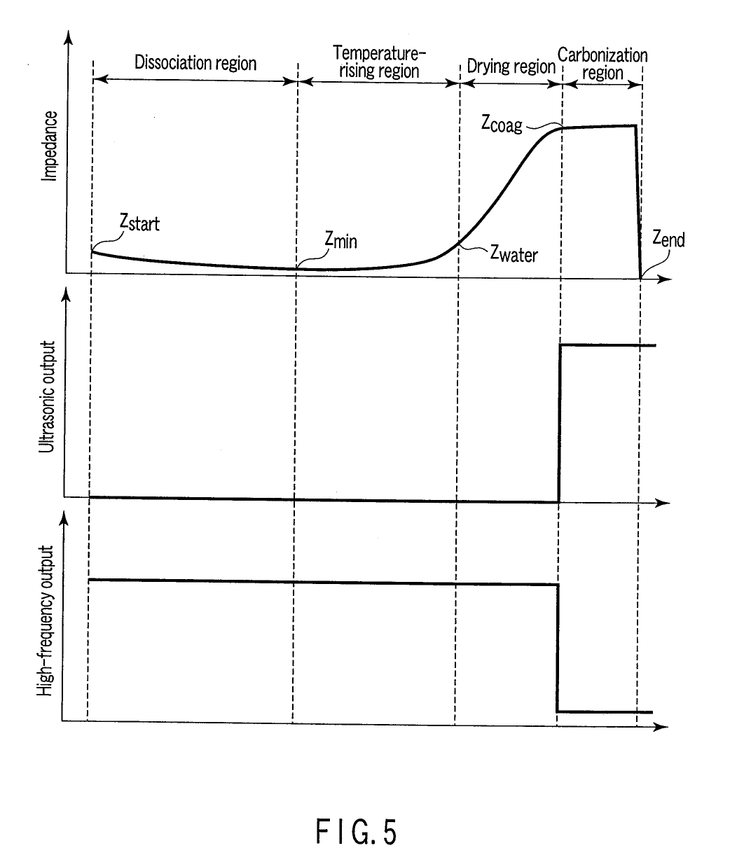

An automatic adjustment method for outputs of the ultrasonic treatment unit and the high-frequency treatment unit by the control module 39 will now be explained with reference to FIGS. 4 and 5.

The automatic adjustment method according to this embodiment avoids insufficient coagulation and burning of the living tissue and realizes rapid incision.

Start of Treatment (S1 and S3)

The living tissue as a treatment target is grasped by the grasping portion 29 of the handpiece 21. Subsequently, the foot switch 34 is turned on. As a result, the food switch detection module 38 detects that the foot switch 34 has been turned on, and the foot switch detection module 38 transmits the foot switch ON signal to the control module 39.

Dissociation Region/Temperature-rising Region/Drying Region (S4 to S7)

The control module 39 that has received the foot switch ON signal transmits the ultrasonic stop signal to the ultrasonic output module 41, and transmits the high-frequency regular output signal to the high-frequency output module 42. The ultrasonic output module 41 that has received the ultrasonic stop signal maintains a stopped state of the ultrasonic treatment unit. The high-frequency output module 42 that has received the high-frequency regular output signal activates the high-frequency treatment unit with the regular output, and the high-frequency current as the regular output flows through the living tissue grasped by the grasping portion 29. The sensor module 43 calculates an impedance of the living tissue grasped by the grasping portion 29, and transmits impedance data to the control module 39. The impedance gradually decreases from the initial value Zstart to the minimum value Zmin, starts rising from the minimum value Zmin and gradually increase, reaches the drying start value Zwater, suddenly increases from the drying start value Zwater, and then stabilizes and takes the coagulation completion value Zcoag. The control module 39 sequentially detects and stores the minimum value Zmin, the drying start value Zwater, and the coagulation completion value Zcoag. Meanwhile, the living tissue coagulates by the high-frequency current, and coagulation of the living tissue is substantially completed when the impedance reaches the coagulation completion value Zcoag.

Carbonization Region (S8 and S9)

When the impedance reaches the coagulation completion value Zcoag, the control module 39 transmits the ultrasonic maximum output signal to the ultrasonic output module 41, and transmits the high-frequency micro output signal to the high-frequency output module 42. The ultrasonic output module 41 that has received the ultrasonic maximum output signal activates the ultrasonic treatment unit with the maximum output, and the living tissue is incised by the ultrasonically vibrated probe. Since the ultrasonic treatment unit is activated with the maximum output that maximizes the incision capability, incision of the living tissue can be rapidly performed. Additionally, since coagulation of the living tissue is completed when incision starts, insufficient coagulation does not occur even if incision is carried out with the maximum output. On the other hand, the high-frequency output module 42 that has received the high-frequency micro output signal activates the high-frequency treatment unit with the micro output, and the micro high-frequency current flows through the living tissue. Since the high-frequency treatment unit is operated with the micro output after completion of coagulation, excess energy is not supplied to the living tissue, and therefore burning of the living tissue is avoided. It is to be noted that the high-frequency treatment unit is not stopped and operated with the micro output in order to measure an impedance of the living tissue.

End of Treatment (S10 and S11)

After end of incision of the living tissue, the foot switch 34 is turned off and the treatment is terminated.

In the surgical operation apparatus according to this embodiment, since the operation of the ultrasonic treatment unit is stopped until coagulation of the living tissue is completed, insufficient coagulation is prevented from occurring in the living tissue. Further, since the ultrasonic treatment unit is operated with the maximum output after coagulation of the living tissue is completed, incision of the living tissue can be rapidly carried out. Furthermore, since the high-frequency treatment unit is operated with the micro output after coagulation of the living tissue is completed, burning can be prevented from occurring in the living tissue.

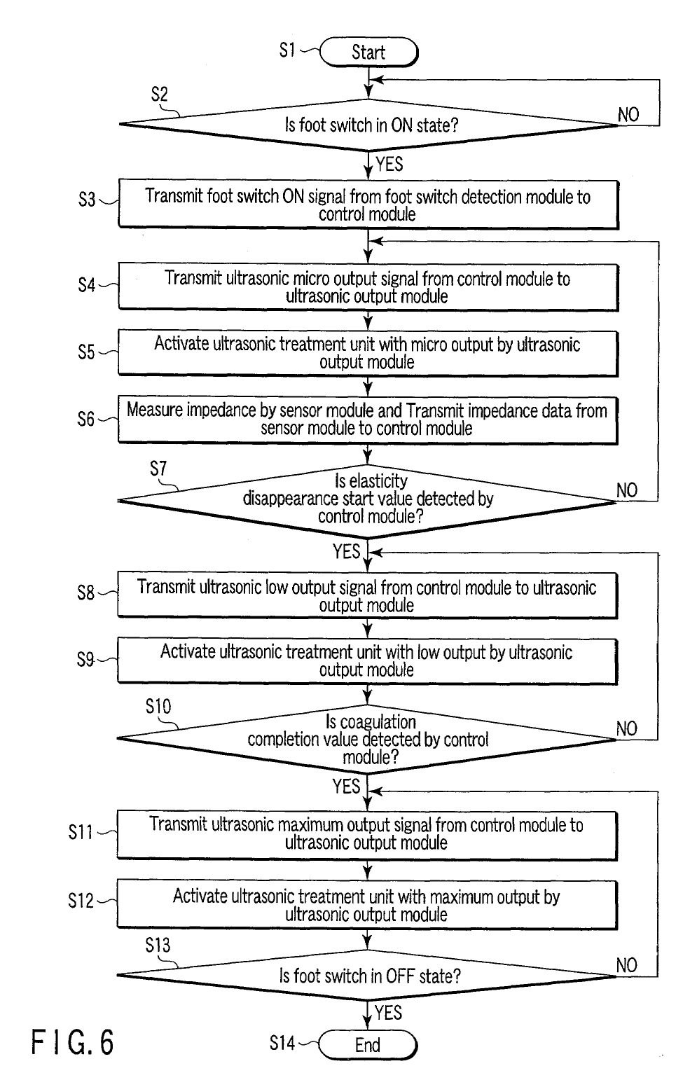

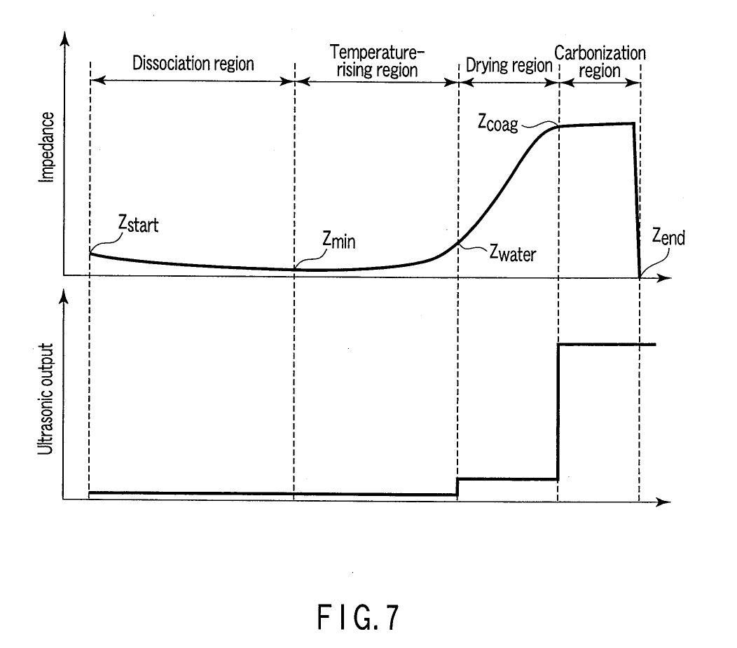

FIGS. 6 and 7 show a second embodiment according to the present invention.

An automatic adjustment method for outputs from the ultrasonic treatment unit and the high-frequency treatment unit by the control module 39 will now be explained with reference to FIGS. 6 and 7.

The automatic adjustment method according to this embodiment avoids slippage of a living tissue from the grasping portion 29 due to ultrasonic vibration. It is to be noted that the automatic adjustment method for the output of the high-frequency unit is the same as that in the first embodiment, thereby omitting an explanation thereof.

Start of Treatment (S1 and S3)

Like the first embodiment, a living tissue as a treatment target is grasped by the grasping portion 29 of the handpiece 21, and the foot switch 34 is turned on.

Dissociation Region/Temperature-Rising Region (S4 to S7)

The control module 39 which has received the foot switch ON signal transmits the ultrasonic micro output signal to the ultrasonic output module 41. The ultrasonic output module 41 that has received the ultrasonic micro output signal activates the ultrasonic treatment unit with the micro output. Here, since the living tissue has elasticity until an impedance reaches the drying start value, i.e., the elasticity disappearance start value Zwater, the living tissue grasped by the grasping portion 29 may possibly slip toward a distal end side by ultrasonic vibration of the probe 26 when the ultrasonic treatment unit is activated with a relatively large output. In this embodiment, since the ultrasonic treatment unit is activated with the micro output until the impedance reaches the elasticity disappearance start value Zwater, slippage of the living tissue is avoided. It is to be noted that the ultrasonic treatment unit may be stopped until the impedance reaches the elasticity disappearance start value Zwater.

Drying Region (S8 to S10)

When the impedance reaches the elasticity disappearance start value Zwater, the control module 39 transmits the ultrasonic low output signal to the ultrasonic output module 41. The ultrasonic output module 41 that has received the ultrasonic low output signal activates the ultrasonic treatment unit with the low output. The impedance is suddenly increased from the drying start value Zwater, then stabilized, and takes the coagulation completion value Zcoag. Meanwhile, since the elasticity of the living tissue disappears and the ultrasonic treatment unit is activated with the low output, the living tissue rarely slips from the grasping portion 29.

Carbonization Region/End of Treatment (S11 to S14)

Like the first embodiment, the ultrasonic treatment unit is activated with the maximum output when an impedance reaches the coagulation completion value Zcoag, and the living tissue is incised. After end of incision of the living tissue, the foot switch 34 is turned off and the treatment is terminated.

In the surgical operation apparatus according to this embodiment, since the ultrasonic treatment unit is activated with the micro output until elasticity of the living tissue starts to disappear, the living tissue grasped by the grasping portion 29 is prevented from slipping toward the distal end side due to ultrasonic vibration of the probe 26. Therefore, since the living tissue dose not have to be again newly grasped and coagulation-incision of the living tissue can be performed by a single operation, an operation time can be reduced and so a burden on an operator can be reduced, and an unnecessary burden is prevented from being imposed on the living tissue.

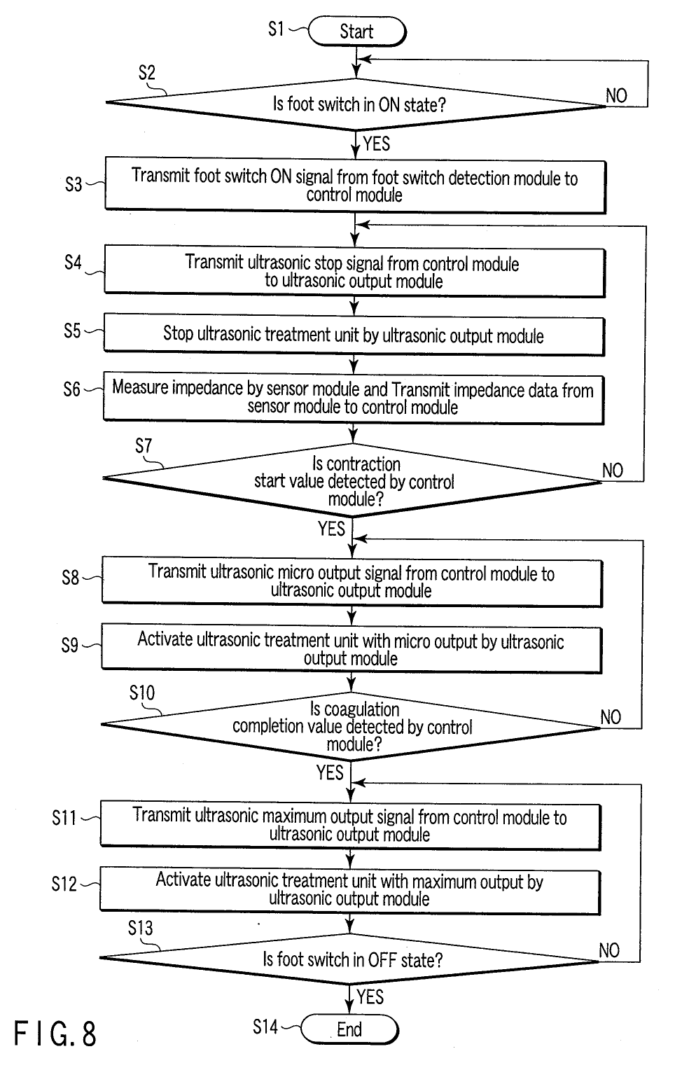

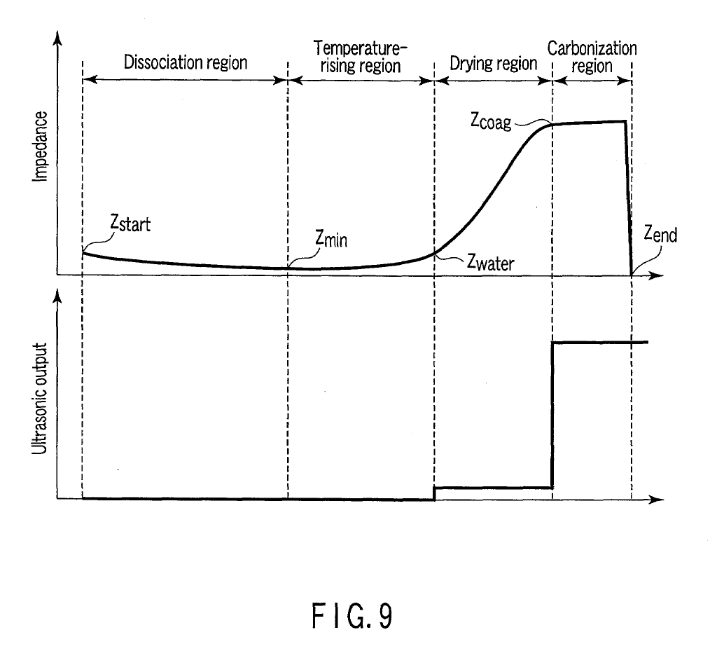

FIGS. 8 and 9 show a third embodiment according to the present invention.

An automatic adjustment method for outputs from the ultrasonic treatment unit and the high-frequency treatment unit by the control module 39 will now be explained with reference to FIGS. 8 and 9.

The automatic adjustment method according to this embodiment avoids adherence of a living tissue to the grasping portion 29 due to contraction of the living tissue. It is to be noted that the automatic adjustment method for the output of the high-frequency treatment unit is the same as that of the first embodiment, thereby omitting an explanation thereof.

Start of Treatment (S1 to S3)

Like the first embodiment, a living tissue as a treatment target is grasped by the grasping portion 29 of the handpiece 21, and the foot switch 34 is turned on.

Dissociation Region/Temperature-Rising Region (S4 to S7)

The control module 39 that has received the foot switch ON signal transmits the ultrasonic stop signal to the ultrasonic output module 41. The ultrasonic output module 41 that has received the ultrasonic stop signal maintains the ultrasonic treatment unit in the stopped state.

Drying Region (S8 to S10)

When the impedance reaches the drying start value, i.e., the contraction start value Zwater, the control module 39 transmits the ultrasonic micro output signal to the ultrasonic output module 41. The ultrasonic output module 41 that has received the ultrasonic micro output signal activates the ultrasonic treatment unit with the micro output. Here, after the impedance reaches the contraction start value Zwater, the living tissue is dried and contracted while being grasped and crushed by the grasping portion 29. If the ultrasonic treatment unit is stopped and the distal end portion of the probe 26 remains stationary, the living tissue is contacted closely with the grasping portion 29 and is deformed in accordance with an outer shape of the grasping portion 29 and so the living tissue may be adhered to the grasping portion 29. In this embodiment, after the impedance reaches the contraction start value Zwater, since the ultrasonic treatment unit is activated with the micro output and the distal end portion of the probe 26 is micro-vibrated, the living tissue deviates from the grasping portion 29, and the living tissue is prevented from being contacted closely with the grasping portion 29, and therefore adherence of the living tissue is prevented. It is to be noted that, since the ultrasonic treatment unit is activated with the micro output, an incision capability becomes substantially zero and the living tissue is not incised before coagulation is completed, and therefore insufficient coagulation of the living tissue is prevented.

Carbonization Region/End of Treatment (S11 to S14)

Like the first embodiment, when the impedance reaches the coagulation completion value Zcoag, the ultrasonic treatment unit is activated with the maximum output, and the living tissue is incised. After end of incision of the living tissue, the foot switch 34 is turned off, and the treatment is terminated.

In the surgical operation apparatus according to this embodiment, since the ultrasonic treatment unit is activated with the micro output after the living tissue starts contraction, the living tissue is prevented from adhering to the grasping portion 29 without insufficient coagulation of the living tissue. Therefore, a complicated operation, i.e., removal of the living tissue that has adhered to the grasping portion 29 is not required, an operation time can be shortened, and a burden on an operator can be reduced.

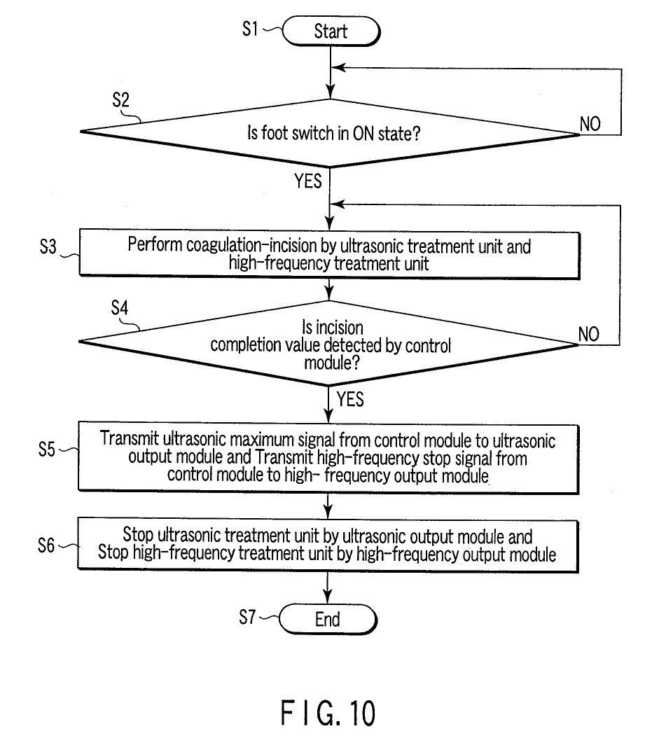

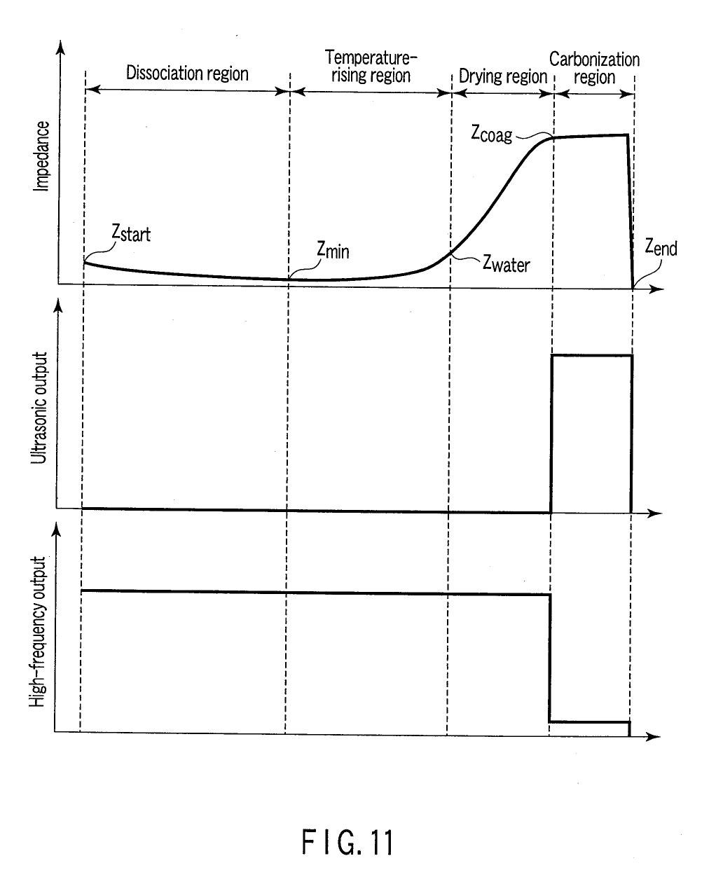

FIGS. 10 and 11 show a fourth embodiment according to the present invention.

An automatic adjustment method according to this embodiment avoids unnecessary outputs from the ultrasonic treatment unit and the high-frequency treatment unit after end of incision.

Start of Treatment/Dissociation Region/Temperature-Rising Region/Drying Region/Carbonization Region (S1 to S4)

Like the first embodiment, a living tissue as a treatment target is grasped by the grasping portion 29 of the handpiece 21, the foot switch 34 is turned on, and coagulation-incision is performed with respect to the living tissue. The impedance varies from the initial value Zstart to the minimum value Zmin, the drying start value Zwater, and the coagulation completion value Zcoag. After the impedance reaches the coagulation completion value Zcoag, the ultrasonic treatment unit is activated with the maximum output to incise the living tissue, the high-frequency treatment unit is activated with the micro output, the micro high-frequency current flows through the living tissue, and measurement of the impedance is continued.

End of Treatment (S5 to S7)

When incision of the living tissue by the probe 26 in the ultrasonic treatment unit is completed and the jaw 27 of the high-frequency treatment unit and the probe 26 come into contact with each other to be short-circuited, the impedance becomes zero, i.e., the incision completion value Zend. When the impedance becomes the incision completion value Zend, the control module 39 outputs the ultrasonic stop signal and the high-frequency stop signal to the ultrasonic output module 41 and the high-frequency output module 42, respectively. The ultrasonic output module 41 that has received the ultrasonic stop signal stops the operation of the ultrasonic treatment unit, and the high-frequency output module 42 that has received the high-frequency stop signal stops the operation of the high-frequency treatment unit. At this time, sound or display may be used to inform an operator that the treatment for the living tissue is completed and the ultrasonic treatment unit and the high-frequency unit cannot operate.

As output restoring conditions of the ultrasonic treatment unit and the high-frequency treatment unit, an output restoring operation, e.g., re-operation of the foot switch 34 is used.

In the surgical operation apparatus according to this embodiment, since the operations of the ultrasonic treatment unit and the high-frequency treatment unit are stopped when incision of the living tissue is completed, unnecessary outputs from the ultrasonic treatment unit and the high-frequency unit are avoided. Therefore, the probe 26 and the jaw 27 is prevented from being excessively worn out due to friction through the ultrasonic vibration of the probe 26 and the probe 26 is prevented from being excessively heated. Furthermore, since the operations of the ultrasonic treatment unit and the high-frequency treatment unit are automatically stopped upon completion of incision, the operation of the surgical operation apparatus is simplified.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A surgical operation apparatus comprising: a handpiece to be held by an operator; and an apparatus main body to be connected with the handpiece,
wherein the handpiece includes: a high-frequency treatment unit to treat a living tissue by a high-frequency current; an ultrasonic treatment unit to treat a living tissue by an ultrasonic vibration; and a sensor unit to perform detection for calculation of an impedance of a living tissue, and
the apparatus main body includes: a high-frequency output module to activate the high-frequency treatment unit; an ultrasonic output module to activate the ultrasonic treatment unit; a sensor module to calculate the impedance of the living tissue based on a detection result obtained from the sensor unit; and a control module to automatically control at least the ultrasonic output module of the high-frequency output module and the ultrasonic output module in accordance with the impedance calculated by the sensor module so as to automatically adjust an output of at least the ultrasonic treatment unit of the high-frequency treatment unit and the ultrasonic treatment unit.

2. The surgical operation apparatus according to claim 1, wherein the control module automatically adjusts an output of the ultrasonic treatment unit, regarding a coagulation completion value indicating that coagulation of the living tissue is completed, such that an output of the ultrasonic treatment unit before the impedance calculated by the sensor module reaches the coagulation completion value becomes smaller than an output of the ultrasonic treatment unit after the impedance calculated by the sensor module reaches the coagulation completion value.

3. The surgical operation apparatus according to claim 2, wherein the control module stops an output of the ultrasonic treatment unit until the impedance calculated by the sensor module reaches the coagulation completion value.

4. The surgical operation apparatus according to claim 2, wherein the control module maximizes an output of the ultrasonic treatment unit after the impedance calculated by the sensor module reaches the coagulation completion value.

5. The surgical operation apparatus according to claim 1, wherein the control module automatically adjusts an output of the high-frequency treatment unit, regarding a coagulation completion value indicating that coagulation of the living tissue is completed, such that an output of the high-frequency treatment unit after the impedance calculated by the sensor module reaches the coagulation completion value becomes smaller than an output of the high-frequency treatment unit before the impedance calculated by the sensor module reaches the coagulation completion value.

6. The surgical operation apparatus according to claim 5, wherein the control module sets an output of the high-frequency treatment unit to a micro output or less after the impedance calculated by the sensor module reaches the coagulation completion value.

7. The surgical operation apparatus according to claim 1, wherein the control module automatically adjusts an output of the ultrasonic treatment unit, regarding an elasticity disappearance start value indicating that elasticity of the living tissue starts to disappear, such that an output of the ultrasonic treatment unit before the impedance calculated by the sensor module reaches the elasticity disappearance start value becomes smaller than an output of the ultrasonic treatment unit after the impedance calculated by the sensor module reaches the elasticity disappearance start value.

8. The surgical operation apparatus according to claim 7, wherein the control module sets an output of the ultrasonic treatment unit to a micro output or less until the impedance calculated by the sensor module reaches the elasticity disappearance start value.

9. The surgical operation apparatus according to claim 1, wherein the control module activate the ultrasonic treatment unit with an output after the impedance calculated by the sensor module reaches a contraction start value indicating that the living tissue starts contraction.

10. The surgical operation apparatus according to claim 9, wherein the control module activates the ultrasonic treatment unit with a micro output after the impedance calculated by the sensor module reaches the contract start value.

11. The surgical operation apparatus according to claim 1, wherein the control module stops output of the high-frequency treatment unit and the ultrasonic treatment unit after the impedance calculated by the sensor module reaches an incision completion value indicating that incision of the living tissue is completed.
